(19) United States
(12) Patent Application Publication
(10) Pub. No.: US 2023/0290470 A1
(43) Pub. Date: Sep. 14, 2023

(54) PRESCRIPTION ADMINISTRATION CART

(71) Applicant: RXSAFE LLC, Vista, CA (US)

(72) Inventor: William K. Holmes, San Diego, CA (US)

(21) Appl. No.: 18/183,745

(22) Filed: Mar. 14, 2023

Related U.S. Application Data
(60) Provisional application No. 63/319,601, filed on Mar. 14, 2022.

(51) Int. Cl. G16H 20/10; G16H 10/60; A61J 1/14

(52) U.S. Cl. CPC G16H 20/10; A61J 1/1437; G16H 10/60

(57) ABSTRACT

Prescription administration system for administering prescriptions to a plurality of patients. One embodiment provides a prescription administration system including a medication tray having a plurality of prescription boxes. Each of the plurality of prescription boxes is configured to receive a plurality of medications corresponding to a single administration time. The prescription administration system also includes an electronic processor. The electronic processor is configured to receive, via a user interface, a selection of an administering unit and identify a prescription box corresponding to the administering unit from the plurality of prescription boxes. The electronic processor is also configured to receive identification information of a patient and determine whether the identification information of the patient corresponds to the prescription box. The electronic processor is further configured to provide an indication when the identification information of the patient corresponds to the prescription box.

### CROSS-REFERENCE TO RELATED APPLICATIONS

This application claims priority to U.S. Provisional Pat. Application No. 63/319,601, filed Mar. 14, 2022, the entire contents of which are incorporated by reference herein.

### FIELD

The present disclosure relates to a prescription administration cart. More particularly, the present disclosure relates to a prescription administration cart used in nursing homes, long term care facilities, and the like.

### SUMMARY

One embodiment provides a prescription administration system including a medication tray having a plurality of prescription boxes. Each of the plurality of prescription boxes is configured to receive a plurality of medications corresponding to a single administration time. The prescription administration system also includes an electronic processor. The electronic processor is configured to receive, via a user interface, a selection of an administering unit and identify a prescription box corresponding to the administering unit from the plurality of prescription boxes. The electronic processor is also configured to receive identification information of a patient and determine whether the identification information of the patient corresponds to the prescription box. The electronic processor is further configured to provide an indication when the identification information of the patient corresponds to the prescription box.

Another embodiment provides a prescription administration cart including a storage area, a medication tray removably received within the storage area, and a plurality of prescription boxes removable received in the medication tray. Each of the plurality of prescription boxes is configured to receive a plurality of medications corresponding to a single administration time.

Another embodiment provides a prescription administration system including a medication tray having a plurality of prescription boxes. Each of the plurality of prescription boxes is configured to receive a plurality of medications corresponding to a single administration time. The prescription administration system also includes an electronic processor. The electronic processor is configured to receive prescription data for a plurality of patients as a group and determine identification information of the medication tray. The electronic processor is also configured to retrieve a portion of the prescription data corresponding to the medication tray and receive a selection of a prescription data unit of the portion of the prescription data corresponding to the medication tray. The electronic processor is further configured to identify a prescription box corresponding to the prescription data unit from the plurality of prescription boxes of the medication tray.

### BRIEF DESCRIPTION OF THE DRAWINGS

FIG. 1 illustrates a prescription administration cart in accordance with some embodiments.

FIG. 2 illustrates a medication tray received within the prescription administration cart of FIG. 1 in accordance with some embodiments.

FIG. 3 illustrates a prescription box received within the medication tray of FIG. 2 in accordance with some embodiments.

FIG. 4 illustrates a pharmacy management system used for filling the medication tray of FIG. 2 in accordance with some embodiments.

FIG. 5 is a block diagram of the prescription administration cart of FIG. 1 in accordance with some embodiments.

FIG. 6 is a block diagram of the medication tray of FIG. 2 in accordance with some embodiments.

FIG. 7 is a block diagram of a pharmacy management system including the filling bay of FIG. 4 in accordance with some embodiments.

FIG. 8 illustrates a flowchart of a method for filling the medication tray of FIG. 2 in accordance with some embodiments.

FIG. 9 illustrates a flowchart of a method for stocking the prescription administration cart of FIG. 1 in accordance with some embodiments.

FIG. 10 illustrates a flowchart of a method for administering medications using the prescription administration cart of FIG. 1 in accordance with some embodiments.

FIG. 11 illustrates an example filling method of FIG. 8 in accordance with some embodiments.

FIG. 12 illustrates an example stocking method of FIG. 9 in accordance with some embodiments.

FIGS. 13 and 14 illustrate an example administering method of FIG. 10 in accordance with some embodiments.

### DETAILED DESCRIPTION

Before any embodiments are explained in detail, it is to be understood that the disclosure is not limited in its application to the details of construction and the arrangement of components set forth in the following description or illustrated in the following drawings. The disclosure is capable of other embodiments and of being practiced or of being carried out in various ways.

Embodiments are also described as methods. However, it is to be understood that all blocks of the method may be optional unless otherwise noted. For example, some blocks of the method may be skipped or altered without deviating from the present disclosure. The blocks of the methods may be performed in any order including the order listed in the methods. Embodiments are illustrated with the help of example figures and related descriptions. The devices, systems, and methods described herein may include more or fewer components than illustrated and described and may perform functions other than those listed herein without deviating from the disclosure.

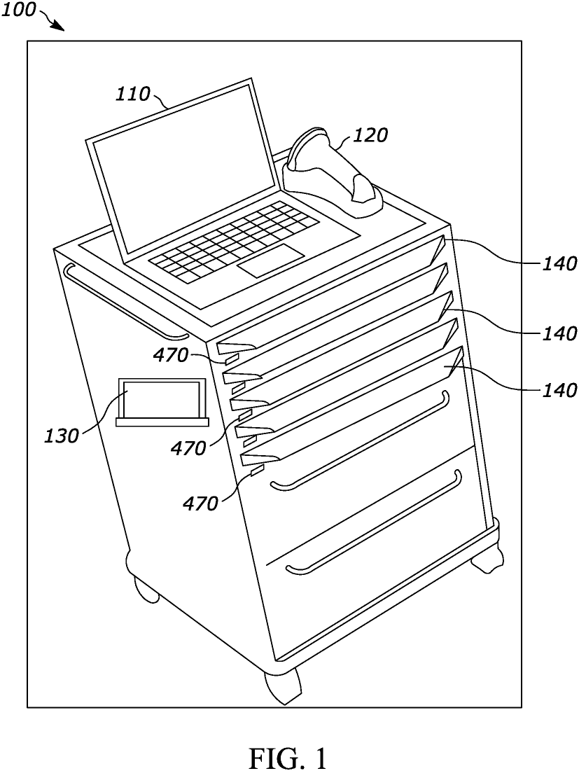

FIG. 1 illustrates an example embodiment of a prescription administration cart 100. The prescription administration cart 100 is used in health care facilities, for example, nursing homes, long-term care facilities, hospitals, and the like to administer prescription medications to patients or residents. The prescription administration cart 100 is communicatively connected with a display device 110, a scanner 120, and a tablet device 130. The prescription administration cart 100 includes a plurality of drawers 140. The prescription administration cart 100 may also include wheels for easy transportation of the prescription administration cart 100 around the health care facility.

The display device 110 is, for example, a laptop computer that is used to display prescription information relating to the patients or residents of the health care facility. The display device 110 may identify each prescription by one or more of a room identification number (or room number) and a patient name or identification number. The scanner 120 is, for example, a bar code or QR code scanner that is used to scan and identify the patients and the prescription medications assigned to the patients. The scanner 120 may alternatively be an RFID tag or NFC tag reader. The tablet device 130 may be used to carry prescription medications to the patients and to provide indications relating to the medication administration.

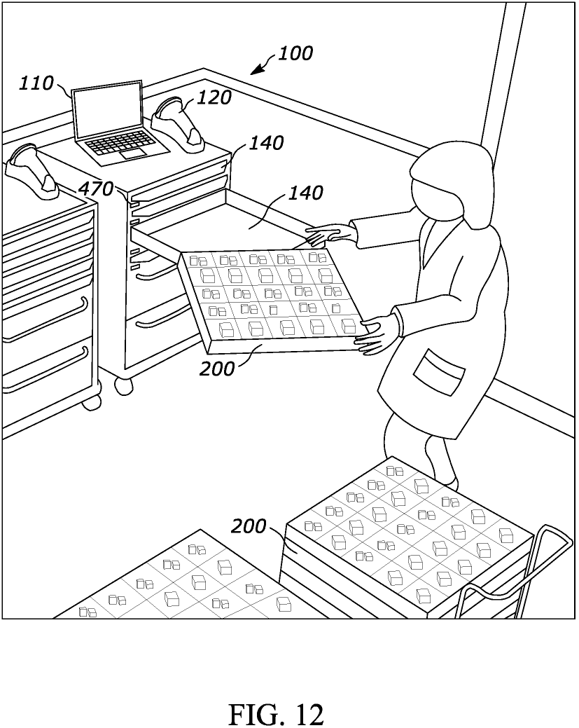

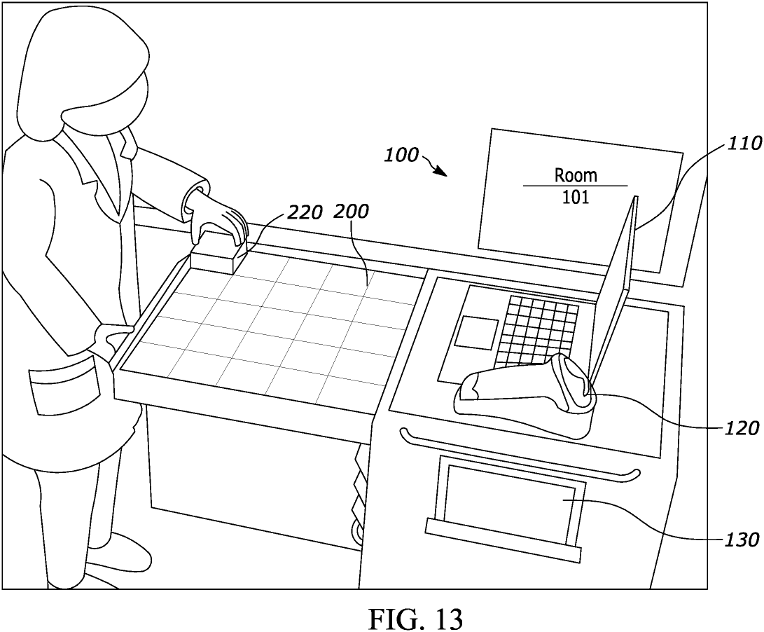

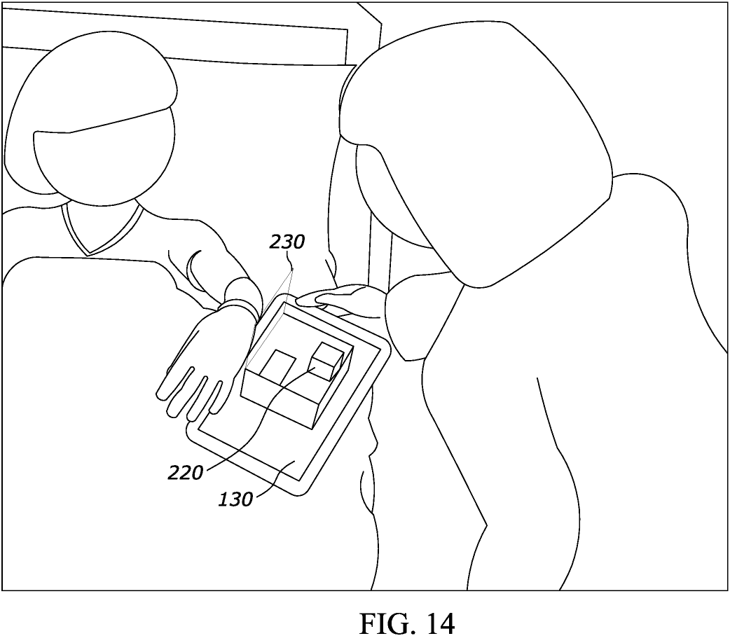

The plurality of drawers 140 provided on the prescription administration cart 100 are configured to receive one or more medication trays 200. Each drawer 140 can be pulled away from the housing of the prescription administration cart 100 (as shown in FIGS. 12 and 13) such that the contents of the medication tray 200 can be accessed by an administrator. As described herein, an administrator is a person (for example, a nurse) that administers the medications to the patient. The drawer 140 can then be pushed back into the housing of the prescription administration cart 100. Each drawer 140 can correspond to a single administration (for example, morning administration, afternoon administration, evening administration, and the like) of the prescription medications within the healthcare facility. The plurality of drawers 140 may be labeled to indicate the timing of administration. Although a plurality of drawers 140 are shown in FIG. 1, the administration cart 100 may include only a single drawer 140 that may receive a single or multiple medication trays 200.

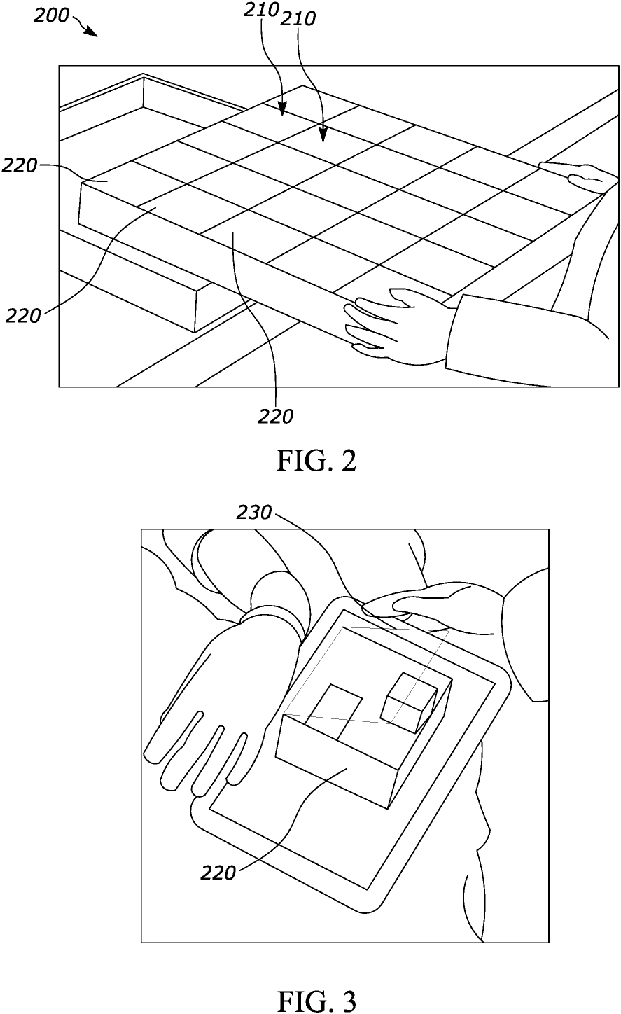

FIG. 2 illustrates an example embodiment of the medication tray 200. The medication tray 200 may include a square, rectangular, hexagonal, or other shape including a base and side walls provided on each side. The medication tray 200 includes a plurality of cubbies 210 provided within the side walls that are configured to receive a plurality of prescription boxes 220. The prescription boxes 220 may be placed and removed from the cubbies 210 from the top of the medication tray 200. The plurality of cubbies 210 are arranged in a grid having a plurality of rows and columns. Each cubby 210 may include a port to connect to a corresponding port of the prescription box 220 received within the cubby 210.

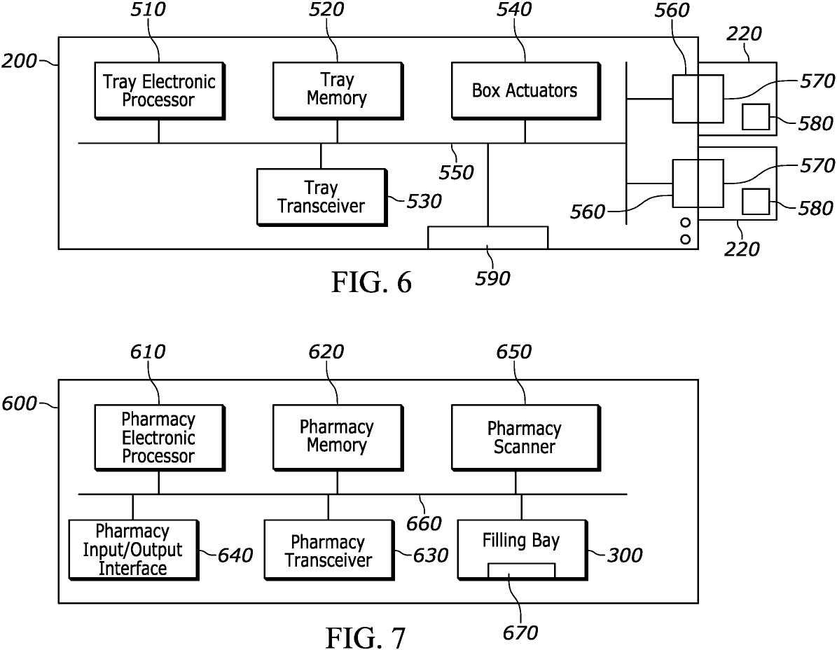

FIG. 3 illustrates an example embodiment of a prescription box 220. The prescription box 220 may include a square, rectangular, hexagonal, or other shape including a base and side walls provided on each side. A box cover 230 is provided on the top of the prescription box 220. The box cover 230 may be actuated using an actuator 580 (shown in FIG. 6) (for example, a motor, a solenoid, a spring-loaded latch, or the like) to be lifted such that the contents of the prescription box 220 may be accessed. In one example, the box cover 230 may be hingedly connected to one side of the prescription box 220. In this example, the actuator 580 raises the box cover 230 along the hinge. Other examples may use other techniques and connections to selectively provide access to the contents of the prescription box 220.

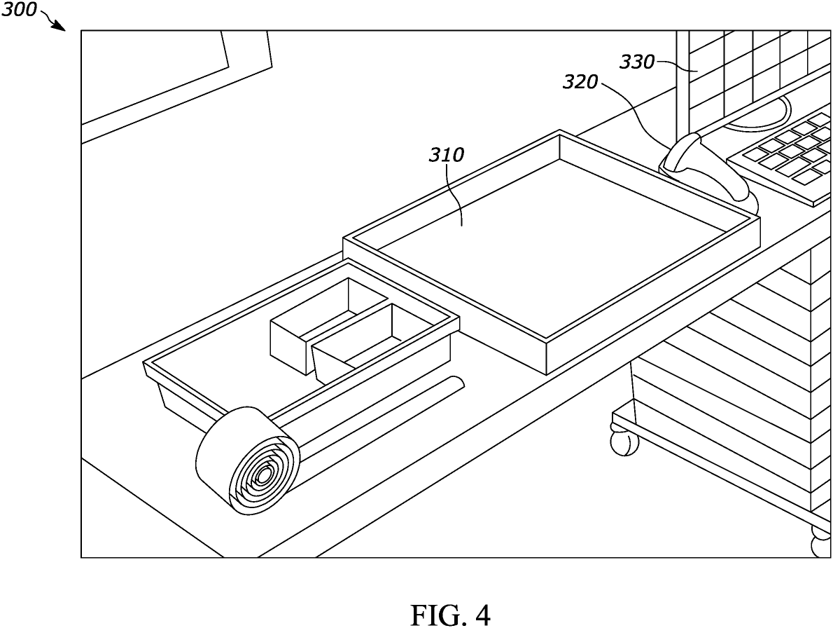

FIG. 4 illustrates an example embodiment of pharmacy management system 300 that is used in a pharmacy to fill the prescription boxes 220 of a medication tray 200. In the example illustrated, the pharmacy management system 300 includes a filling bay 310, a pharmacy scanner 320, and a display device 330. The filling bay 310 may include a similar shape as the medication tray 200 with larger dimensions such that medication tray 200 may be received within the filling bay 310. The filling bay 310 may include a port that connects to a corresponding port of the medication tray 200 to facilitate communication between the medication tray 200, the prescription boxes 220, and the pharmacy management system 300. As described above, the prescription boxes 220 are communicatively connected to the medication tray 200 using ports. In some embodiments, rather than ports, the medication tray 200, the prescription boxes 220, and the pharmacy management system 300 may be communicatively connected using a wireless connection.

The pharmacy scanner 320 is, for example, a bar code scanner or a QR code scanner similar to the scanner 120. The pharmacy scanner 320 may be used to identify the medication trays 200 that are to be filled and the medications that are filled in the prescription boxes 220. The display device 330 may be a separate computing device or a display monitor connected to the pharmacy management system 300. The display device 330 displays the prescription information, patient information, and medication information to assist the pharmacist or pharmacy technician in packaging the pharmaceuticals within the medication trays 200.

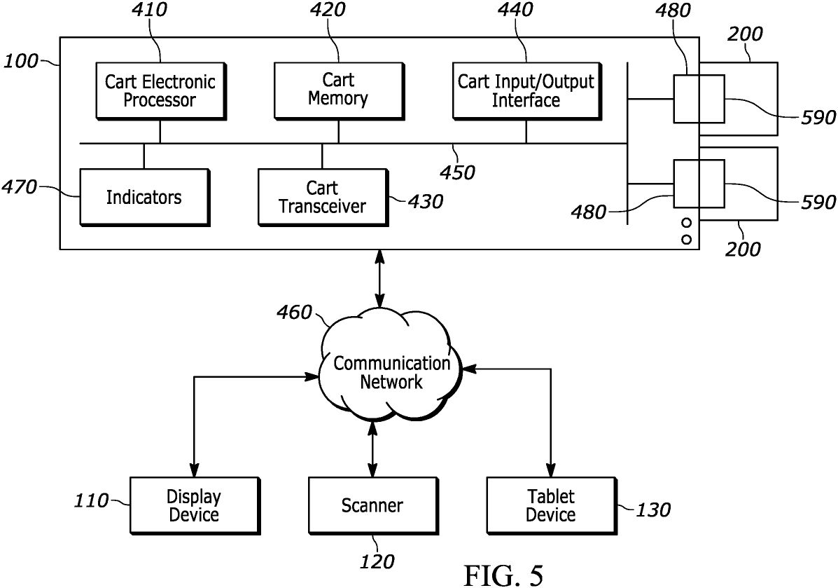

FIG. 5 is a simplified block diagram of the prescription administration cart 100. In the example illustrated, the prescription administration cart 100 includes a cart electronic processor 410, a cart memory 420, a cart transceiver 430, and a cart input/output interface 440. The cart electronic processor 410, the cart memory 420, the cart transceiver 430, and the cart input/output interface 440 communicate over one or more control and/or data buses (for example, a cart communication bus 450). The prescription administration cart 100 communicates with the peripheral devices including the display device 110, the scanner 120, and the tablet device 130 over a communication network 460. In some embodiments, the communication network 460 may include a long-range communication network, for example, the Internet, a cellular network, and the like. In other embodiments, the communication network 460 includes a short-range communication network, for example, a Wi-Fi® network, a Bluetooth® network, and the like.

In some embodiments, the cart electronic processor 410 is implemented as a microprocessor with separate memory, such as the cart memory 420. In other embodiments, the cart electronic processor 410 may be implemented as a microcontroller (with cart memory 420 on the same chip). In other embodiments, the cart electronic processor 410 may be implemented using multiple processors. In addition, the cart electronic processor 410 may be implemented partially or entirely as, for example, a field programmable gate array (FPGA), an applications-specific integrated circuit (ASIC), and the like and the cart memory 420 may not be needed or may be modified accordingly. In the example illustrated, the cart memory 420 includes nontransitory, computer-readable memory that stores instructions that are received and executed by the cart electronic processor 410 to carry out the functionality of the prescription administration cart 100 described herein. The cart memory 420 may include, for example, a program storage area and a data storage area. The program storage area and the data storage area may include combinations of different types of memory, such as read-only memory, and random-access memory. In some embodiments, the prescription administration cart 100 may include one cart electronic processor 410, and/or plurality of cart electronic processors 410, for example, in a cluster arrangement, one or more of which may be executing none, all, or a portion of the applications of the prescription administration cart 100 described below, sequentially or in parallel across the one or more cart electronic processors 410. The one or more cart electronic processors 410 comprising the prescription administration cart 100 may be geographically colocated or may be geographically separated and interconnected vial electrical and/or optical interconnects. For example, cart electronic processor 410 may be distributed either fully or partially in one or more of the peripheral devices (that is, the display device 110, the scanner 120, and the tablet device 130) of the prescription administration cart 100. One or more proxy servers or load balancing servers may control which one or more cart electronic processors 410 perform any part or all of the applications provided below.

The cart transceiver 430 enables wired and/or wireless communication between the prescription administration cart 100 and one or more of the display device 110, the scanner 120, the tablet device 130, the medication trays 200, and the prescription boxes 220. In some embodiments, the cart transceiver 430 may comprise separate transmitting and receiving components, for example, a transmitter and a receiver. The cart input/output interface 440 may include one or more input mechanisms (for example, a touch pad, a keypad, and the like), one or more output mechanisms (for example, a display, a speaker, and the like). In some embodiments, the display device 110 acts as the cart input/output interface 440 or as an extension of the cart input/output interface 440. In some embodiments, the cart input/output interface 440 includes indicators 470 (for example, LED indicators) provided on each of the plurality of drawers 140 of the prescription administration cart 100.

In some embodiments, the prescription administration cart 100 includes a plurality of cart ports 480 provided in the plurality of drawers 140. The plurality of cart ports 480 are configured to be mechanically and electrically connected to a communication port 590 of the medication tray 200. In these embodiments, the components of the medication tray 200 including the prescription boxes 220 are communicatively coupled to the prescription administration cart 100 over the cart communication bus 450. Accordingly, the cart electronic processor 410 may be used to control a box actuator 540 of the medication tray 200 or the actuators 580 of the prescription boxes 220. In other embodiments, the medication tray 200 and the prescription administration cart 100 may be wirelessly connected.

FIG. 6 is a simplified block diagram of the medication tray 200. In the example illustrated, the medication tray 200 includes a tray electronic processor 510, a tray memory 520, a tray transceiver 530, and box actuators 540. The tray electronic processor 510, the tray memory 520, the tray transceiver 530, and the box actuators 540 communicate over one or more control and/or data buses (for example, a tray communication bus 550). The tray electronic processor 510, the tray memory 520 and the tray transceiver 530 may be implemented similar to the cart electronic processor 410, the cart memory 420, and the cart transceiver 430. As described above, the medication trays 200 receive a plurality of prescription boxes 220. The box actuators 540 include, for example, a motor, a solenoid and the like that move a prescription box 220 received in a cubby 210 so that the prescription box 220 may be easily removed from the cubby 210.

In some embodiments, the medication tray 200 includes a plurality of tray ports 560 provided in the plurality of cubbies 210. The plurality of tray ports 560 are configured to be mechanically and electrically connected to box ports 570 of the prescription box 220. In these embodiments, the components of the prescription box 220 are communicatively coupled to the medication tray 200 over the tray communication bus 550. Accordingly, the tray electronic processor 510 may be used to control an actuator 580 (for example, a motor, a solenoid, a spring-loaded latch, and the like) of the prescription box 220 that actuates the box cover 230 of the prescription box 220. In other embodiments, the prescription box 220 and the actuator 580 may be wirelessly connected with the medication tray 200. In some embodiments, the medication tray 200 also includes a communication port 590 for connection to the filling bay 310 or the prescription administration cart 100.

In some embodiments, the prescription box 220 may include a separate electronic processor, memory, and transceiver. In some embodiments, the tray electronic processor 510, the tray memory 520, and the tray transceiver 530 may be distributed between the medication tray 200 and the prescription boxes 220.

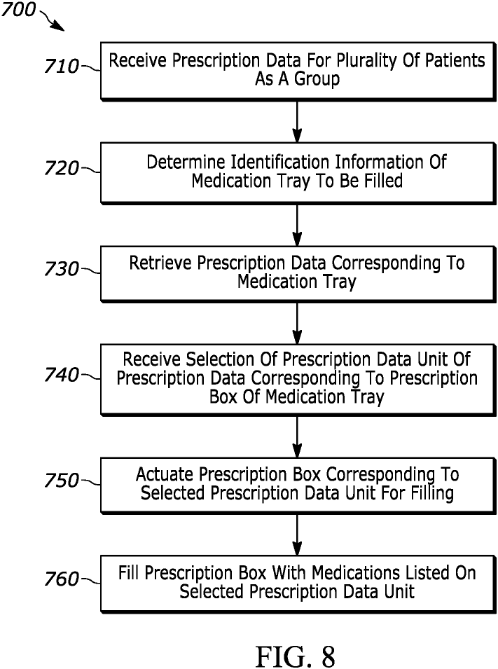

FIG. 7 is a simplified block diagram of the pharmacy management system 300 that is provided in pharmacy to fill the medication trays 200. The pharmacy management system 300 includes a pharmacy electronic processor 610, a pharmacy memory 620, a pharmacy transceiver 630, a pharmacy input/output interface 640 (for example, the display device 330), the filling bay 310, the pharmacy scanner 320. The pharmacy electronic processor 610, the pharmacy memory 620, the pharmacy transceiver 630, the pharmacy input/output interface 640, the filling bay 310, and the pharmacy scanner 320 communicate over one or more control and/or data buses (for example, a pharmacy communication bus 650). The pharmacy electronic processor 610, the pharmacy memory 620, the pharmacy transceiver 630, and the pharmacy input/output interface 640 may be implemented similar to the cart electronic processor 410, the cart memory 420, the cart transceiver 430, and the cart input/output interface 440. In some embodiments, the filling bay 310 includes a bay port 660 mechanically and electrically connected to the communication port 590 of the medication tray 200.

A healthcare facility may include several wards or wings per floor. Each ward or wing of each floor may be divided into a unit. The healthcare facility may use a plurality of prescription administration carts 100 with each prescription administration cart 100 of the plurality of prescription administration carts 100 being used for a single unit. The medication trays 200 received within the prescription administration cart 100 for the single unit may include all the prescription medications for the patients within the unit. The medications may be prescribed by a healthcare provider located at the healthcare facility or at a different facility. The prescriptions are sent to a pharmacy for filling. The pharmacy may be a part of the healthcare facility or may be an unaffiliated facility.

FIG. 8 is a flowchart of an example method 700 for filling a medication tray 200. The method 700 may be implemented by the pharmacy management system 300. In the example illustrated, the method 700 includes receiving prescription data for a plurality of patients as a group (at block 710). The prescription data may be received from the healthcare facility or from a healthcare provider consulting with the patients of the healthcare facility. The prescription data is received as a group for the plurality of patients of the healthcare facility. The prescription data may be divided between units and medication trays 200. For example, prescription data may be divided between several pages, with each page corresponding to one medication tray 200. Each page may include prescription data for one administration (for example, a morning administration, an afternoon administration, an evening administration, and the like) for one group of patients. The group of patients may be a plurality of patients located closely (for example, within a single unit) of the healthcare facility.

The method 700 also includes determining, using the pharmacy scanner 320, identification information of a medication tray 200 to be filled (at block 720). Empty medications trays 200 are received by the pharmacy from the healthcare facility. The medication trays 200 are individually placed on the filling bay 310 for filling by a pharmacist or pharmacy technician. When a medication tray 200 is ready to be filled, the pharmacist scans the medication tray 200. For example, the pharmacist uses the pharmacy scanner 320 to scan a bar code or QR code of the medication tray 200. The pharmacy electronic processor 610 determines the identification information of the medication tray 200 based on the scanned bar code or QR code. The pharmacy electronic processor 610 may consult a database of medication trays 200 to determine the identification information based on the scanned information.

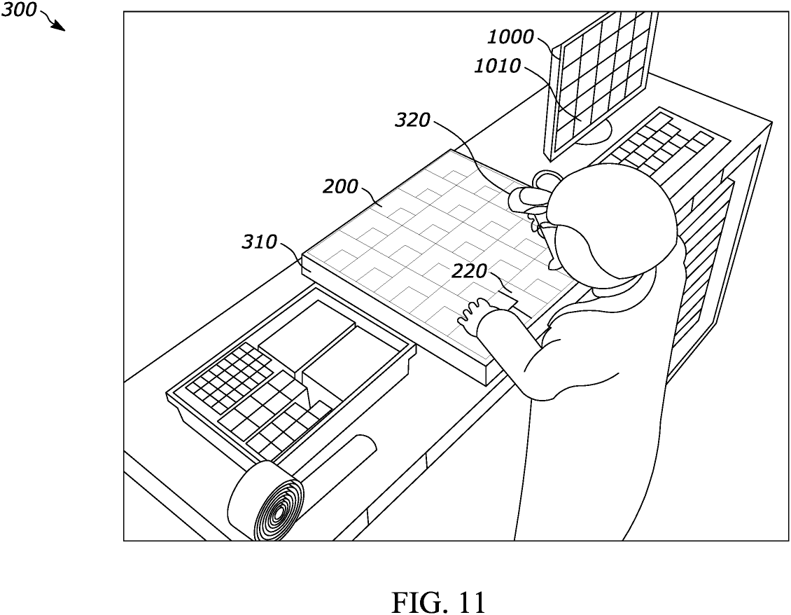

The method 700 includes retrieving, using the pharmacy electronic processor 610, prescription data corresponding to the medication tray 200 (at block 730). Prior to or after scanning and determining the identification information of the medication tray 200, the medication tray 200 may be placed on the filling bay 310. As discussed above, the prescription data may be grouped into pages, with each page corresponding to a medication tray 200. The pharmacy electronic processor 610 retrieves the page corresponding to the medication tray 200 on the filling bay 310. The prescription information page may be displayed on a user interface (for example, the display device 330) of the pharmacy management system 300. For example, as shown in FIG. 11, the prescription page may be displayed on a display monitor 1000 of the pharmacy management system 300.

The method 700 also includes receiving, via a user interface, a selection of a prescription data unit of the prescription data corresponding to a prescription box 220 of the medication tray 200 (at block 740). As discussed above, the prescription data corresponding to the medication tray 200 may be displayed. The prescription data includes a plurality of prescription data units, with each data unit corresponding to each prescription box 220 of the medication tray 200. The prescription data may be displayed such that the prescription data units are arranged in a grid including rows and columns that match the arrangement of the prescription boxes 220 in the medication tray 200. As shown in FIG. 11, the pharmacist may then select individual prescription data units by clicking on or touching the portion of the display 1000 corresponding to the prescription data unit 1010.

The method 700 includes actuating, using the actuator 580, the prescription box 220 corresponding to the selected prescription data unit for filling (at block 750). The pharmacy electronic processor 610 or the tray electronic processor 510 determines the prescription box 220 corresponding the selected prescription data unit. A control signal may be sent to the actuator 580 of the prescription box 220 to open the box cover 230 of the prescription box 220. The prescription box 220 may be actuated in response to receiving the selection of the prescription data unit. In some embodiments, rather than using an actuator 580 to open the prescription box 220, the pharmacist may manually open the prescription box 220 for filling based on an indication provided by the pharmacy management system 300.

The method 700 next includes filling the prescription box 220 with the medications listed on the selected prescription data unit (at block 760). The pharmacist may scan the prescribed medication using the pharmacy scanner 320 and place the medication in the prescription box 220. In some embodiments, the prescribed medication may be in pre-packaged pouch or vial. In other embodiments, unpackaged pills or medications may be placed in the prescription box 220. Once the prescription box 220 is filled, the pharmacist may manually close the box cover 230 of the prescription box 220. In some embodiments, the prescription box 220 may be automatically closed using the actuator 580 when filling is complete or when the selection for a next prescription data unit is received. In the example shown in FIG. 11, the pharmacist has selected the second prescription box 220 and uses the prescription information to fill the second prescription box 220. The method 700 including blocks 740 to 760 may be repeated until the desired number of prescription boxes 220 in the medication tray 200 are filled. The method 700 including block 720 to 760 may be repeated until a desired number of medication trays 200 are filled. Once the medication trays 200 are filled, the medication trays 200 are transported to the healthcare facility for administration.

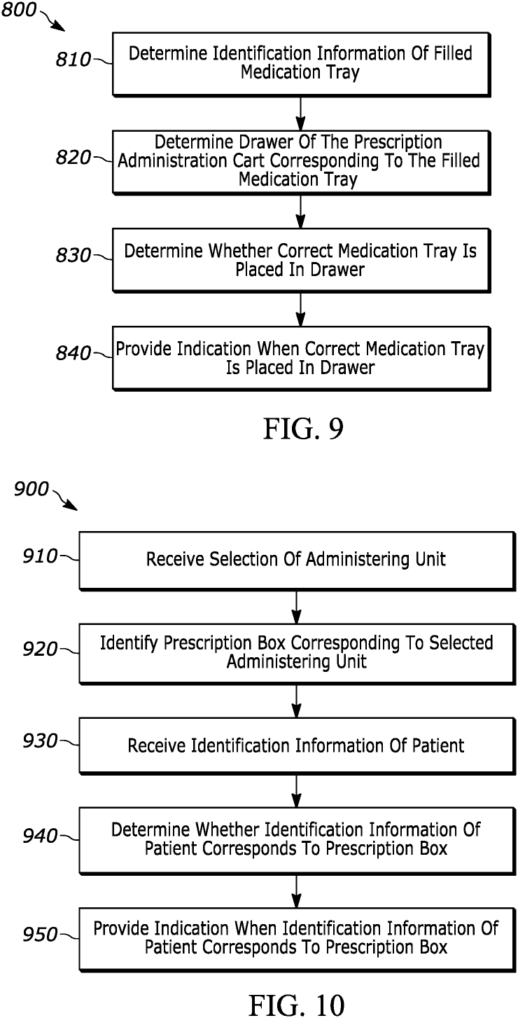

FIG. 9 is a flowchart of an example method 800 for stocking prescription administration carts 100. The method 800 may be implemented by the prescription administration carts 100. In the example illustrated, the method 800 includes determining, using the scanner 120, identification information of a filled medication tray 200 (at block 810). Filled medication trays 200 are received by the healthcare facility from the pharmacy. The administrator at the healthcare facility may scan the medication tray 200. For example, the administrator uses the scanner 120 to scan a bar code or QR code of the medication tray 200. The cart electronic processor 410 determines the identification information of the medication tray 200 based on the scanned bar code or QR code. The cart electronic processor 410 may consult a database of medication trays 200 to determine the identification information based on the scanned information.

The method 800 includes determining, using the cart electronic processor 410, a drawer 140 of the prescription administration cart 100 corresponding to the filled medication tray 200 (at block 820). The drawer 140 may be determined based on the identification information of the medication tray 200. In some embodiments, an indicator 470 corresponding to the identified drawer 140 may be activated in response to determining the drawer 140 corresponding to the filled medication tray 400. For example, as shown in FIG. 12, when the medication tray 200 corresponds to the third drawer 140, the cart electronic processor 410 may activate an indicator 470 corresponding to third drawer 140.

The method 800 also includes determining, using the cart electronic processor 410, whether a correct medication tray 200 is placed in the drawer 140 (at block 830). When the administrator places the medication tray 200 in the drawer 140, the medication tray 200 may send identification information of the medication tray 200 to the prescription administration cart 100 via the ports 480 and 590 or over a wireless connection to the prescription administration cart 100. Based on the identification information, the cart electronic processor 410 may determine whether the correct medication tray 200 is placed in the drawer 140.

The method 800 includes providing, using the indicators 470, an indication when the correct medication tray 200 is placed in the drawer 140 (at block 840). The cart electronic processor 410 may activate an indicator 470 corresponding to the drawer 140 when the correct medication tray 200 is placed in the drawer 140. The indicator 470 may be, for example, an LED. In some embodiments, an indication having a first color (for example, green) may be provided when the correct medication tray 200 is placed in the drawer 140. In some embodiments, a second indication having a second color (e.g., red) may be provided when an incorrect medication tray 200 is placed in the drawer 140. As shown in the example of FIG. 12, the indicators 470 are illuminated when the administrator places the correct medication tray 200 in the corresponding drawer 140. The method 800 may be repeated until all medication trays 200 are placed into the prescription administration cart 100 or until all prescription administration carts 100 are filled.

The prescription administration carts 100 are used by administrators (for example, nurses) to administer prescription medications to patients. The administrators transport the prescription administration carts 100 to the patients’ rooms, retrieve the corresponding medications, and administer the medications to the patients. FIG. 10 illustrates a flowchart of an example method 900 of administering medications using the prescription administration cart 100. The method 900 may be implemented by the prescription administration cart 100. In the example illustrated, the method 900 includes receiving, via a user interface of the prescription administration cart 100, a selection of an administering unit (at block 910). An administering unit is, for example, a selection of a patient, a room of the patient, a particular administering time (for example, morning, afternoon, evening, and the like) for a patient, and the like. The selection may be received on the display device 110. Specifically, the administrator selects the patient or room on the display device 110 when the prescription cart is near the patient or the room. In the example shown in FIG. 13, the administrator may select room no. 101 on the display device 110. In some embodiments, the administrator may select the patient or room by scanning, for example, a barcode or QR code on a wall plate or placard associated with the patient or room. In other embodiments, the patient or room may be automatically selected using GPS, NFC, or the like when the prescription administration cart 100 is moved within a set distance of the patient or room.

The method 900 also includes identifying, using the cart electronic processor 410, a prescription box 220 corresponding to the selected administering unit (at block 920). The cart electronic processor 410 may associate each prescription box 220 loaded into the prescription administration cart 100 with an administering unit. When the corresponding administering unit is selected by the administrator, the cart electronic processor 410 identifies the prescription box 220 corresponding the administering unit selected. The cart electronic processor 410 may provide an indication of the identified prescription box 220. For example, an indication may be provided on the display device 110 showing a location of the prescription box 220 in the drawer 140 and/or in the medication trays 200 in the drawers 140. In some embodiments, the cart electronic processor 410 may activate the indicator 470 corresponding to the drawer 140 in which the prescription box 220 is located. Additional or alternative indications may also be provided, for example, using indicators on the medication tray 200 or the prescription box 220. In some embodiments, the cart electronic processor 410 may provide a control signal to the medication tray 200 to control the box actuator 540 to actuate the prescription box 220. When the prescription box 220 is actuated, the prescription box 220 may extend from the medication tray 200 to be retrieved by the administrator. For example, as shown in FIG. 13, the first prescription box 220 is extended when the pharmacist selects the first administering unit corresponding to the first prescription box 220 on the display device 110.

When the prescription box 220 is retrieved by the administrator, the administrator may place the prescription box 220 on the tablet device 130 and take the tablet device 130 to the patient as shown in FIG. 14. The method 900 further includes receiving, using the cart electronic processor 410, identification information of a patient (at block 930). In some embodiments, the administrator uses the scanner 120 to scan a wristband or other wearable device of a patient to receive the identification information of the patient. In other embodiments, as shown in FIG. 14, the administrator may place the prescription box 220 and/or the tablet device 130 in close proximity to the patient’s wrist band or other wearable device to receive the identification information. In these embodiments, an RFID, Bluetooth® low-energy, or the like systems may be used to obtain identification information.

The method 900 includes determining, using the cart electronic processor 410, whether the identification information of the patient corresponds to the prescription box 220 (at block 940). The cart electronic processor 410 includes prescription information for each patient. The cart electronic processor 410 may also receive information relating to which prescription box 220 includes the administering unit for each patient. The cart electronic processor 410 may retrieve the prescription information for the patient and determine whether the medications in the prescription box 220 match the prescription information.

The method 900 includes providing an indication when the identification information of the patient corresponds to the prescription box 220 (at block 950). When the cart electronic processor 410 determines that the identification information of the patient corresponds to or matches the prescription box 220, the cart electronic processor 410 may provide an indication on the tablet device 130. For example, the tablet device 130 may show a first color (for example, green) when the identification information matches the prescription box 220. In some embodiments, the cart electronic processor 410 may provide a control signal to the prescription box 220 to actuate the box cover 230 in response to determining that the identification information of the patient corresponds to the prescription box 220. A second indication may be provided when the identification information does not correspond to the prescription box 220. For example, the tablet device 130 may show a second color (for example, red) when the identification information does not match the prescription box 220. The cart electronic processor 410 may provide a control signal (or may provide no signal) to the prescription box 220 to prevent opening of the box cover 230 when the identification information does not correspond to the prescription box 220.

1. A prescription administration system comprising:
a medication tray including a plurality of prescription boxes, each of the plurality of prescription boxes configured to receive a plurality of medications corresponding to a single administration time; and
an electronic processor configured to
receive, via a user interface, a selection of an administering unit,
identify a prescription box corresponding to the administering unit from the plurality of prescription boxes,
receive identification information of a patient,
determine whether the identification information of the patient corresponds to the prescription box, and
provide an indication when the identification information of the patient corresponds to the prescription box.

2. The prescription administration system of claim 1, further comprising:
a prescription administration cart including a plurality of storage areas and a plurality of medication trays configured to receive the plurality of prescription boxes, wherein the medication tray is one of the plurality of medication trays, and wherein the plurality of medication trays is removably received in the plurality of storage areas.

3. The prescription administration system of claim 2, wherein the prescription administration cart includes a plurality of drawers, and wherein the plurality of drawers define the plurality of storage areas.

4. The prescription administration system of claim 1, further comprising a tablet device communicatively coupled to the electronic processor, wherein the indication is provided using the tablet device.

5. The prescription administration system of claim 1, further comprising a scanner configured to scan a wearable device of the patient, wherein the identification information of the patient is received by scanning, using the scanner, the wearable device.

6. The prescription administration system of claim 1, wherein the indication is a first indication, wherein the electronic processor is further configured to
provide a second indication when the identification information of the patient does not correspond to the prescription box.

7. A prescription administration cart comprising:
a storage area;
a medication tray removably received within the storage area; and
a plurality of prescription boxes removable received in the medication tray, each of the plurality of prescription boxes configured to receive a plurality of medications corresponding to a single administration time.

8. The prescription administration cart of claim 7, further comprising:
an electronic processor configured to
receive, via a user interface, a selection of an administering unit,
identify a prescription box corresponding to the administering unit from the plurality of prescription boxes,
receive identification information of a patient,
determine whether the identification information of the patient corresponds to the prescription box, and
provide an indication when the identification information of the patient corresponds to the prescription box.

9. The prescription administration cart of claim 8, further comprising a tablet device communicatively coupled to the electronic processor, wherein the indication is provided using the tablet device.

10. The prescription administration cart of claim 8, further comprising a scanner communicatively coupled to the electronic processor and configured to scan a wearable device of the patient, wherein the identification information of the patient is received by scanning, using the scanner, the wearable device.

11. The prescription administration cart of claim 8, wherein the indication is a first indication, wherein the electronic processor is further configured to
provide a second indication when the identification information of the patient does not correspond to the prescription box.

12. The prescription administration cart of claim 7, further comprising:
an electronic processor configured to
receive prescription data for a plurality of patients as a group,
determine identification information of the medication tray,
retrieve a portion of the prescription data corresponding to the medication tray,
receive a selection of a prescription data unit of the portion of the prescription data corresponding to the medication tray, and
identify a prescription box corresponding to the prescription data unit from the plurality of prescription boxes of the medication tray.

13. The prescription administration cart of claim 12, further comprising:
a plurality of storage areas, wherein the storage area is one of the plurality of storage areas;
a plurality of medication trays removably received within the plurality of storage areas, wherein the medication tray is one of the plurality of medication trays,
wherein the electronic processor is further configured to
determine whether the medication tray is placed in the storage area after the medication tray is filled, and
provide an indication when the medication tray is placed in the storage area.

14. The prescription administration cart of claim 7, further comprising a drawer that defines the storage area.

15. A prescription administration system comprising:
a medication tray including a plurality of prescription boxes, each of the plurality of prescription boxes configured to receive a plurality of medications corresponding to a single administration time; and
an electronic processor configured to
receive prescription data for a plurality of patients as a group,
determine identification information of the medication tray,
retrieve a portion of the prescription data corresponding to the medication tray,
receive a selection of a prescription data unit of the portion of the prescription data corresponding to the medication tray, and
identify a prescription box corresponding to the prescription data unit from the plurality of prescription boxes of the medication tray.

16. The prescription administration system of claim 15, further comprising:
a prescription administration cart including a plurality of medication trays, wherein the medication tray is one of the plurality of medication trays,
wherein the electronic processor is further configured to
identify a storage area of the prescription administration cart corresponding to the medication tray when the medication tray is filled,
determine whether the medication tray is placed in the storage area in response to identifying the storage area, and
provide an indication when the medication tray is placed in the storage area.

17. The prescription administration system of claim 16, further comprising an indicator provided on the storage area, wherein the indication is provided using the indicator of the storage area.

18. The prescription administration system of claim 16, wherein the prescription administration cart includes a drawer that defines the storage area.

19. The prescription administration system of claim 15, wherein the electronic processor is further configured to actuate the prescription box in response to identifying the prescription box.

20. The prescription administration system of claim 15, wherein the prescription data unit includes information related to a single administration unit provided to a single patient at a particular time of day.
